(19) United States
(12) Patent Application Publication
(10) Pub. No.: US 2008/0103448 A1
(43) Pub. Date: May 1, 2008

(54) Stylet Having a Roughened Outer Surface

(76) Inventor: Greg M. Schorn, Milford, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/559,689

(22) Filed: Nov. 14, 2006

Related U.S. Application Data
(60) Provisional application No. 60/863,508, filed on Oct. 30, 2006.

(51) Int. Cl. A61M 5/178

(52) U.S. Cl. 604/164.01

(57) ABSTRACT

A stylet has a proximal end a distal end and an outer surface. The stylet is substantially cylindrical. The outer surface of the stylet is treated, or roughened, preferably by a glass peening or a bead blasting operation, such that its maximum profile peak height is greater than 30 μm, its roughness average is greater than 5 μm, and its root mean square roughness is greater than 8 μm. This results in reducing the removal force of the treated stylet from a silicone catheter.

### FIELD OF THE INVENTION

### Background of the Invention

The use of a stiffening member or stylet within a flexible catheter to stiffen the catheter to help guide the introduction of the catheter into the desired location in a patient is well known. Conventionally once the catheter is in place, the stylet is removed and discarded.

Catheters are often made of low durometer silicone. For example, ventricular catheters are often made of low durometer (about 50 A) silicone, and have a predetermined outer diameter, inner diameter and length. Such a ventricular catheter is often provided preloaded with a polished stainless steel round stylet. However, neurosurgeons have frequently complained that they experience great difficulty when removing the stylet from the catheter due to friction between the stylet and catheter, which can cause, among other things, displacement of the distal end of the catheter from the target site, and/or cutting of the catheter.

To reduce the friction, attempts have been made to coat the stylet with PTFE or modify the geometrical shape so that it has a square cross-section. Others still have tried to modify the manufacturing process to decrease the amount of so called stickiness of the catheter by either including a post curing process or compounding the catheter with a filler, such as barium sulfate.

However, surgeons have been generally dissatisfied with these attempts as they are accustomed to a conventional catheter feel and design. For example, surgeons have complained that a square-shaped stylet does not provide them with the same placement feel as a conventional round stylet.

Accordingly, there remains a need for a stylet that can be preloaded in a conventional catheter, such as, for example, a low durometer catheter, and can provide sufficient stiffness to the catheter to assist a user in guiding the catheter to its desired location in a patient while thereafter permitting the stylet to be easily removed from the catheter.

It is also an aspect of the invention to provide a stylet design that is simple to produce and that can be made of standard materials by convenient and cost effective procedures.

In light of the present disclosure and the practice of the present invention, other advantages and solutions to other problems will become apparent to those skilled in the relevant art.

### SUMMARY OF THE INVENTION

The present invention generally provides a stylet that has a proximal end, a distal end and an outer surface. The stylet is substantially cylindrical. The outer surface of the stylet is treated, or roughened, preferably by a glass peening or a bead blasting operation, such that its maximum profile peak height is greater than 30 μm, its roughness average is greater than 5 μm, and its root mean square roughness is greater than 8 μm. This results in reducing the removal force of the treated stylet from a silicone catheter.

### BRIEF DESCRIPTION OF THE DRAWINGS

The invention will be more fully understood from the following detailed description taken in conjunction with the accompanying drawings, in which:

FIG. 1 is a side view of a test method for removing a stylet from a catheter, according to the present invention;

FIG. 2 is a cross-sectional view of the stylet only taken along lines 2-2 of FIG. 1 and looking in the direction of the arrows;

FIG. 3 is a chart illustrating the stylet removal force of a conventional stylet and a roughened stylet according to the present invention; and

FIG. 4 is a chart showing various roughness properties of a conventional stylet and a roughened stylet according with the present invention.

### DETAILED DESCRIPTION OF THE INVENTION

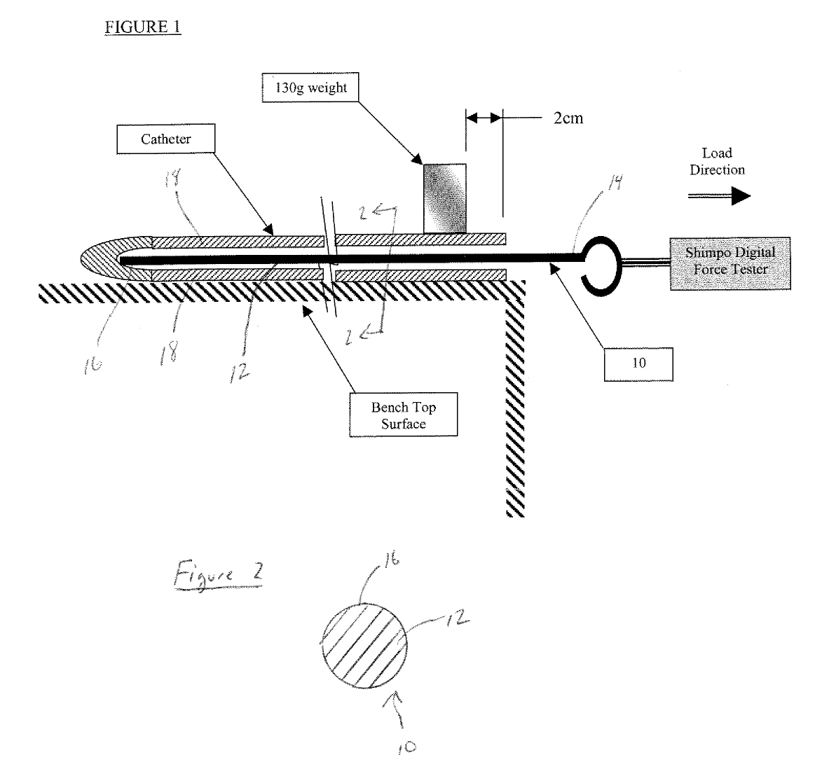

Referring now to FIGS. 1 and 2, the present invention generally provides a stylet 10 that has an elongate stylet body 12 having a proximal end 14, a distal end and an outer surface 16. For the sake of clarity in the drawings, only the cross-section of the stylet is shown in FIG. 2. The stylet is substantially cylindrical at least for a majority of its length, and preferably for its entire length. Outer surface 16 is treated, or roughened, preferably by a glass peening or a bead blasting operation, such that its maximum profile peak height is greater than 30 μm, its roughness average is greater than 5 μm, and its root mean square roughness is greater than 8 μm.

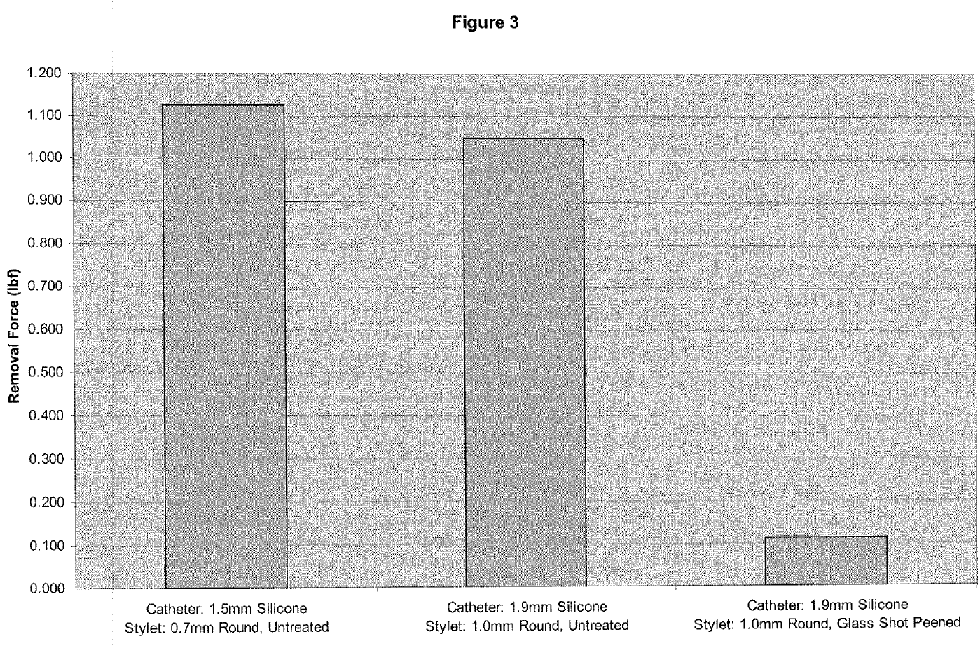

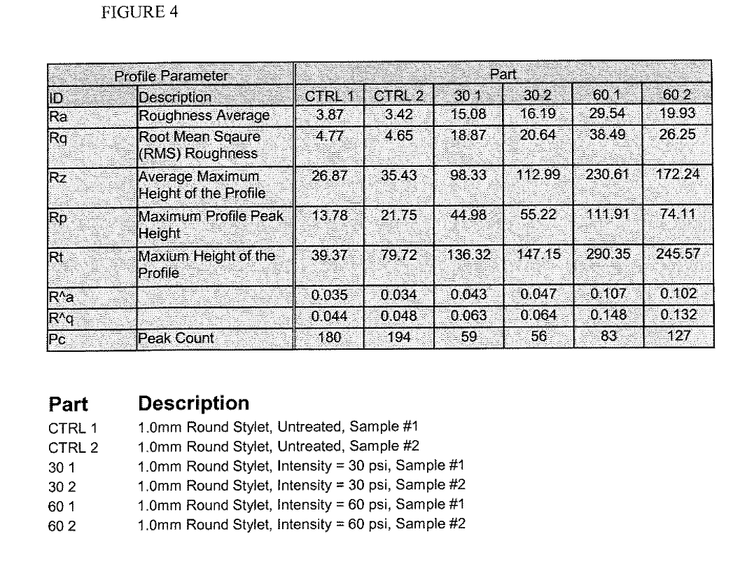

Surface treatment is typically provided by conventional shot peening. As one skilled in the art is readily aware, in shot peening, metal or glass shot is bombarded against the surface of the component with suitable intensity and overlapping coverage. The conventional use for shot peening is to reduce the potential for stress cracking on metal parts by creating a compressed layer at the surface. The present inventors use the shot peening process to roughen the outer surface of the stylet. U.S. Pat. No. 5,057,108 issued to Shetty et al. discloses a process of stainless steel shot blasting, glass bead blasting, electropolishing, and passivation. In addition, a method of surface finishing a medical device shield using metallic media is described in U.S. Pat. No. 5,673,473 to Johnson, et al. The disclosures of these two U.S. Patents are hereby fully incorporated by reference in their entirety. In a currently preferred exemplary embodiment, stylet 10 is subject to a glass shot peening for at least 10 minutes with a glass shot size of about 100 μm and with an intensity range between 30-60 psi. This results in the stylet 10 being essentially 100% treated along the majority of the stylet's length. The test results shown in FIGS. 3 and 4 included stylets treated in this manner.

Stylet 10 is preferably preloaded in a low durometer catheter 18. When preloaded, the stylet provides sufficient stiffness to the catheter to assist a user in guiding the catheter to its desired location in a patient. Thereafter, because the outer surface of stylet 10 has been roughened, stylet 10 is easily removed from the catheter 18 by a user. In a currently preferred exemplary embodiment, stylet 10 is made of stainless steel, and catheter 18 is made of silicone.

Referring now to FIG. 1, a test method is illustrated, which was used to compare the removal force of a catheter according to the present invention as compared to a conventional stainless steel polished round stylet. In accordance with this test method, the following steps are followed:
1. Place catheter with loaded stylet onto a flat bench top surface 20 such that the proximal end of the catheter extends over the edge of the table.
2. Place a 130 g weight approximately 2 cm from the proximal end of the catheter. (This is to consistently hold the catheter in place during the test without compressing the ID against the stylet.)
3. Connect a digital force gauge to the stylet (e.g., a Shimpo Digital Force Tester, 0-10 N)
4. Tare the force gauge and set for peak hold; and
5. Extract the stylet from the catheter and record the peak load required to remove it from the catheter.

Following this test procedure, the stylet removal force according to the present invention for a 1.0 mm round stylet is about 0.1 lbf, whereas for a conventional round stylet, which is 0.7 mm round or 1.0 mm round, the removal force is about 1.1 lbf as shown on FIG. 3. Thus, the removal force is about a magnitude of order less when using a stylet that has been treated in accordance with the present invention.

Referring now to FIG. 4, a chart showing various roughness properties of a conventional stylet (CTRL 1 and CTRL 2) and a roughened stylet in accordance with the present invention (30 1, 30 2, 60 1, and 60 2). The properties were measured in accordance with ANSI B46.1: 1985 and ISO 4287: 1996.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A stylet comprising:
an elongate stylet body having a proximal end, a distal end and an outer surface, said stylet being substantially cylindrical at least for a majority of its length, said outer surface being treated such that its maximum profile peak height is greater than 30 μm.

2. The stylet of claim 1, wherein the stylet body is substantially cylindrical for essentially its entire length.

3. The device of claim 2, wherein the stylet body is made of stainless steel.

4. A stylet comprising:
an elongate stylet body having a proximal end, a distal end and an outer surface, said stylet being substantially cylindrical at least for a majority of its length, said outer surface being treated such that its roughness average is greater than 5 μm.

5. A stylet comprising:
an elongate stylet body having a proximal end, a distal end and an outer surface, said stylet being substantially cylindrical at least for a majority of its length, said outer surface being treated such that its root mean square roughness is greater than 8 μm.

6. A stylet comprising:
an elongate stylet body having a proximal end, a distal end and an outer surface, said stylet being substantially cylindrical at least for a majority of its length, said outer surface being treated such that the stylet has a removal force from a catheter of less than 0.8 lbf.

7. The stylet of claim 6, wherein the removal force is about 0.1 lbf.

8. A method of removing a stylet from a catheter, wherein the catheter is made of an elastomeric material, and the stylet is made of a rigid material, the stylet having an elongate stylet body having a proximal end, a distal end and an outer surface, said stylet being substantially cylindrical at least for a majority of its length, said outer surface being treated such that its outer surface is roughened and its maximum profile peak height is greater than 30 μm; the method comprising the steps of:
guiding a stylet loaded catheter to a desired target; and
removing the stylet.

9. A method of removing a stylet from a catheter, wherein the catheter is made of an elastomeric material, and the stylet is made of a rigid material, the stylet having an elongate stylet body having a proximal end, a distal end and an outer surface, said stylet being substantially cylindrical at least for a majority of its length, said outer surface being treated such that its outer surface is roughened and its roughness average is greater than 5 μm; the method comprising the steps of:
guiding a stylet loaded catheter to a desired target; and
removing the stylet.

10. A method of removing a stylet from a catheter, wherein the catheter is made of an elastomeric material, and the stylet is made of a rigid material, the stylet having an elongate stylet body having a proximal end, a distal end and an outer surface, said stylet being substantially cylindrical at least for a majority of its length, said outer surface being treated such that its outer surface is roughened and its root mean square roughness is greater than 8 μm; the method comprising the steps of:
guiding a stylet loaded catheter to a desired target; and
removing the stylet.

11. A method of removing a stylet from a catheter, wherein the catheter is made of an elastomeric material, and the stylet is made of a rigid material, the stylet having an elongate stylet body having a proximal end, a distal end and an outer surface, said stylet being substantially cylindrical at least for a majority of its length, said outer surface being treated such that its outer surface is roughened and it has a removal force from a catheter of less than 0.8 lbf, the method comprising the steps of:
guiding a stylet loaded catheter to a desired target; and
removing the stylet.

12. A method of treating a stylet so that it can be easily removed from a catheter, wherein the catheter is made of an elastomeric material, and the stylet is made of a rigid material, the stylet having an elongate stylet body having a proximal end, a distal end and an outer surface, said stylet being substantially cylindrical at least for a majority of its length, the method comprising the steps of:
subjecting the stylet body to a glass shot peening process.

13. The method of treating a stylet according to claim 12, wherein the subjecting step achieves essentially 100% coverage along the majority of the stylet's length.

14. The method of treating a stylet according to claim 12, wherein the subjecting step continues for at least 10 minutes with a glass shot size of about 100 μm and with an intensity range between 30-60 psi.

15. A method of treating a stylet so that it can be easily removed from a catheter, wherein the catheter is made of an elastomeric material, and the stylet is made of a rigid material, the stylet having an elongate stylet body having a proximal end, a distal end and an outer surface, said stylet being substantially cylindrical at least for a majority of its length, the method comprising the steps of:
subjecting the stylet body to a glass shot peening or bead blasting process.
